(19) United States
(12) Patent Application Publication
(10) Pub. No.: US 2019/0388280 A1
(43) Pub. Date: Dec. 26, 2019

(54) POST-SURGERY COMPRESSION GARMENT

(71) Applicant: THE MARENA GROUP, LLC, Lawrenceville, GA (US)

(72) Inventors: Jeffrey Dan STEPHENS, Atlanta, GA (US); Linda Marie Burhance, Newtown, CT (US)

(21) Appl. No.: 16/433,899

(22) Filed: Jun. 6, 2019

Related U.S. Application Data
(60) Provisional application No. 62/681,451, filed on Jun. 6, 2018.

(51) Int. Cl. A61F 13/14; A61F 5/03

(52) U.S. Cl. CPC A61F 13/143; A61F 2013/00536; A61F 5/03

(57) ABSTRACT

A compression garment for use after cardiothoracic surgery, comprising a body panel configured to lie in proximity to an upper region of a patient and formed from a compression material, and including a front side adjacent to a front upper region of the wearer, and a rear side configured to lie adjacent to a rear upper region of the patient. Further included is a shoulder strap associated with the rear side of the body panel and configured to extend away from the rear side and configured to removably associate with the front side of the body panel, the shoulder strap is adjustable to adjust the extension of the shoulder strap. The garment also includes a pillow formed in the front side, the pillow having a generally oblong shape and sized to project inwardly towards the patient, the pillow configured to apply pressure to the sternal area.

### CROSS-REFERENCE TO RELATED APPLICATION

This application claims the benefit under 35 U.S.C. § 119(e) of U.S. Provisional Patent Application No. 62/681,451, filed Jun. 6, 2018, the disclosure of which is incorporated by reference in its entirety.

### FIELD

The present disclosure relates, in exemplary embodiments, to a compression garment. More specifically, the present disclosure relates, in exemplary embodiments, to a compression garment for post-surgery use.

### BACKGROUND

The invasive nature of cardiothoracic surgery demands increased recovery time due, in most part, to the sternotomy and the extended length of time over which the sternum heals. As such, the daily activities of the patient may slow the healing process due to the pressure placed on the sternum as a result of patient movement, breathing, and/or coughing. Ultimately, this pressure may cause stress on the sternal wires and/or stitching holding the patient's chest cavity closed, thereby slowing the healing process and potentially resulting in a reopening of the chest cavity. It would be desirable for a patient to have a garment that, when worn, could apply localized pressure to the sternal wound area.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to the more detailed description below.

Generally described, the present disclosure provides in a first exemplary embodiment a compression garment for use after cardiothoracic surgery, comprising a body panel configured to lie in proximity to an upper region of a patient and formed from a compression material, and including a front side adjacent to a front upper region of the wearer, and a rear side configured to lie adjacent to a rear upper region of the patient. Further included is a shoulder strap associated with the rear side of the body panel and configured to extend away from the rear side and configured to removably associate with the front side of the body panel, the shoulder strap is adjustable to adjust the extension of the shoulder strap. The garment also includes a pillow formed in the front side, the pillow having a generally oblong shape and sized to project inwardly towards the patient, the pillow configured to apply pressure to the sternal area.

Other features will become apparent upon reading the following detailed description of certain exemplary embodiments, when taken in conjunction with the appended claims.

### BRIEF DESCRIPTION OF THE DRAWINGS

The drawings disclose exemplary embodiments in which like reference characters designate the same or similar parts throughout the figures, of which:

FIG. 1 is an perspective view of one exemplary embodiment of the present disclosure;

FIG. 2 is an exploded view of a pillow illustratively included in the exemplary embodiment of FIG. 1; and,

FIG. 3 is a perspective view of the exemplary embodiment of FIG. 1 and showing one illustrative position of an exemplary embodiment on a patient.

### DETAILED DESCRIPTION

Unless otherwise indicated, the drawings are intended to be read (for example, cross-hatching, arrangement of parts, proportion, degree, or the like) together with the specification, and are to be considered a portion of the entire written description of this invention. As used in the following description, the terms “horizontal”, “vertical”, “left”, “right”, “up” and “down”, “upper” and “lower” as well as adjectival and adverbial derivatives thereof (for example, “horizontally”, “upwardly”, or the like), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate.

FIG. 1 shows one exemplary embodiment of a garment 12 in accordance with the present disclosure is adapted to be worn by a post-operative patient 10 following cardiothoracic or other surgery to provide sternum support and protect the patient 10 from being injured during recovery. Illustratively, the garment 12 is configured to be worn by a patient 10 following surgery due to the invasive nature of the surgery and the extended healing process following a sternotomy (a surgical procedure whereby a patient's cardiothoracic region 20 is cut and the sternum is then cracked/split to gain access to the heart). The garment 12, therefore, is configured to decrease the risk of the patient's chest cavity reopening, and to increase the rate of healing. It will be appreciated by one skilled in the art that the garment 12 may also be used following a myriad of types of operations or procedures resulting in a need for consistently applied pressure upon the post-operative patient 10.

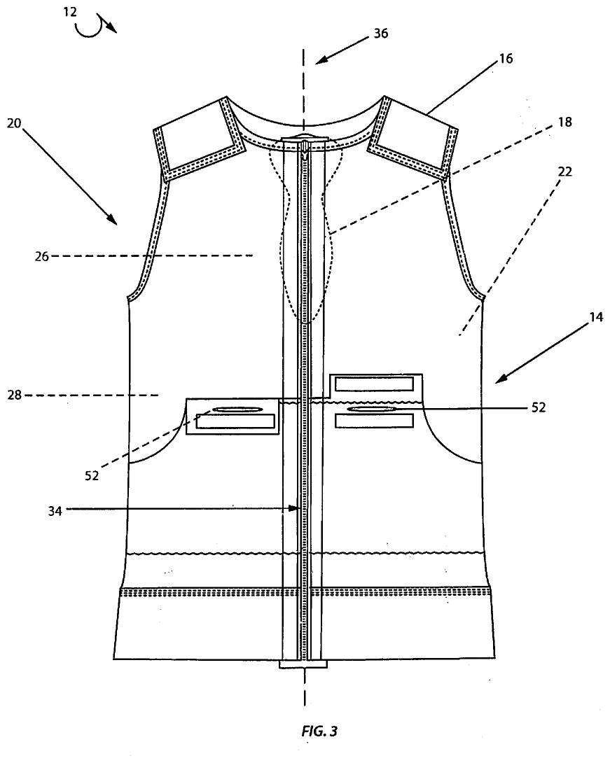

As shown in FIG. 1, in one exemplary embodiment, a compression garment 12 includes a body panel 14, a shoulder strap 16 coupled to a body panel 14, and a pillow 18 integrally formed in the body panel 14. The pillow 18 is configured to engage an upper region 20 of the patient 10 as shown in FIGS. 1 and 3. The body panel 14 is configured to lie in close proximity to the upper region 20 of the patient 10 and includes a front side 22, a rear side 24, and a lateral side 28 at which the front side 22 and the rear side 24 meet and couple. The shoulder strap 16 is coupled to the rear side 24 and configured to removably couple to the front side 22. In one exemplary embodiment, the pillow 18 is integrally formed in the front side 22 and configured to translate the compression of the body panel 14 to the patient 10. In exemplary embodiments the pillow 18 is generally oblong in shape. In exemplary embodiments the pillow 18 has an outline generally approximating the outline of a human sternum. Illustratively, the pillow 18 is generally sternal-shaped and configured to engage the upper region 20 of the patient 10 such that the pillow 18 applies pressure upon the sternal wires/stitching when the garment 12 is in use.

The garment 12 is further configured to tightly secure the upper region 20 of the patient 10 when in use such that a front upper region 26 of the patient 10 and a rear upper region (not shown) of the patient 10 are simultaneously engaged. In exemplary embodiments, the body panel 14 may also include a side seam 32 integrally formed in the lateral side 28. In exemplary embodiments, the body panel 14 includes two lateral sides 28 configured to engage a side body portion (not shown) of the patient 10 and couple the front side 22 and rear side 24, thereby forming the body panel 14 from a singular piece of material. Further, the garment 12 may include side seams 32 integrally formed in both lateral sides 28. In exemplary embodiments, the body panel 14 is formed from at least one resiliently elastic material such that the body panel 14 is form-fitting. In exemplary embodiments, the body panel 14is further formed by side seams 32 into the lateral sides 28. The garment 12 is can be made in different sizes so as to be configured to accommodate patients 10 ranging in weight and body mass distribution.

The side seams 32 are configured to release a portion of the compression applied on the upper region 20 of the patient 10 by permitting some of the force from the elasticity of the body panel 14 to be distributed away from the patient 10, thereby allowing patients with increased visceral fat to use the garment 12 and do so more comfortably. Illustratively, the resiliently elastic material is formed from fabric having a 4-way stretch and configured to apply a calculated compression value appropriate to each size type of patient 10 so that the compression is consistent and comfortable for each patient 10. The calculated compression value may be determined based on various factors such as a patient's weight, height, body mass distribution, and other factors that, when taken in the aggregate, result in a compression value unique to each patient 10. The compression characteristics are substantially maintained throughout the time the patient 10 wears the garment 12. This is true whether the patient 10 is sedentary or active as the form-fitting material is configured to maintain constant pressure on the patient 10 such that the pillow 18 remains in constant contact with the upper region 20 of the patient 10.

In using the garment 12, the post-operative patient 10 does not have not have to use other commonly known methods of applying pressure upon her/his upper region 20. Further, the garment 12 can reduce or eliminate problems associated with using a cardiac pillow alone, such as the patient's noncompliance or reflexive movements, patient's with dementia, or the like. This places the mind of the patient 10, a caregiver, and a patient's family more at ease as there is less personal responsibility required of the patient 10 to comply with proper use of the garment 12. As such, until the garment 12 is removed from the patient 10, the garment 12 will maintain constant pressure on the patient's upper region 20.

Illustratively, the garment 12 is removably put on by the patient 10 to provide the aforementioned pressure on the upper region 20 of the patient 10 as shown in FIGS. 1 and 3. In coupling the garment 12 to the patient 10 (i.e., putting the garment on the patient), the caregiver first opens the front side 22 of the body panel 14 via a closure mechanism 34. In one exemplary embodiment, the closure mechanism 34 is a zipper as shown in FIGS. 1 and 3 and configured to extend along a central axis 36 from an area proximate to or at the upper edge 38 of the front side 22 to an area proximate to or at the bottom edge 40 of the front side 22. Other closure mechanisms 34 known in the art may also be used, such as, but not limited to, hook and loop, eye hook, snaps, buttons, or the like. Upon releasing the closure mechanism 34 (i.e., unzipping where a zipper is used), the caregiver may now position the garment 12 on the post-operative patient 10 such that the rear side 24 of the body panel 14 is placed adjacent to an upper back 42 of the patient 10. The front side 22, the rear side 24, and the lateral side(s) 28 cooperate to form an aperture 44 configured to receive an arm (not shown) of the patient 10. Upon placing the patient's upper back 42 in contact with and adjacent to the rear side 24 of the body panel 14, the caregiver then guides the patient's arm through the aperture 44. In exemplary embodiments, the body panel 14 forms two apertures 44, each aperture 44 configured to receive an arm of the patient 10.

The garment 12 is now positioned on the patient 10 with the front upper region 20 facing away from the rear side 24 of the body panel 14 and the patient's arms extending through the apertures 44. The caregiver may then engage the closure mechanism 34 and secure the garment 12 on the patient 10. In assuring proper positioning of the pillow 18, the caregiver should confirm that the pillow 18 is positioned on the front upper region 20 of the patient 10 prior to securing the garment 12. Proper placement of the pillow 18 varies between patients 10 due to body mass composition, anatomical features, the patient's unique incision, and other factors. As such, the pillow 18 is deemed properly placed when the desired calculated compression is applied on the stitching/sternal wires proximate to the patient's incision. Illustratively, the incision extends along the sternum/breastbone of the patient 10 in a generally vertical manner in relation to a central axis 36.

In exemplary embodiments, the body panel 14 of the garment 12 includes a front side 22, a rear side 24, and a lateral side 28 formed from a continuous piece of fabric such that the body panel 14 is uninterrupted as shown in FIGS. 1 and 3. In other exemplary embodiments, a plurality of pieces or panels of fabric may be used which are attached to each other to form the body panel 14. The front side 22 extends from an area proximate to or at the upper edge 38 of the front side 22 to an area proximate to or at the bottom edge 40 of the body panel 14. The rear side 24 extends from an area proximate to or at an upper edge 42 of the front rear side 24 to an area proximate to or at the bottom edge 40 of the body panel 14. The front side 22 and the rear side 24 are sized similarly to each other and shaped such that the upper edge 38 of the front side 22 and the upper edge 42 of the rear side 24 are, illustratively, arcuately shaped. The upper edge 38 of the front side 22 is configured to removably couple to the shoulder straps 16. The upper edge 42 of the rear side 24 is configured to removably couple to the shoulder straps 16.

In exemplary embodiments, the front side 22 of the body panel 14 may include a pocket 46, as shown in FIG. 3. Illustratively, the pocket 46 may be attached to the front side 22 by stitching at an attachment point 48 extending generally perpendicular to the central axis 36, but other methods commonly known in the practice of garment assembly may also be used. The pocket 46 may be formed to include an opening 50 illustratively located above the attachment point 48 on the front side 22, therebetween forming an opening 50 into the pocket 46. The pocket 46 is sized to receive various devices and objects, and, as such, the size of the pocket 46 may vary. Placement of the pocket may also vary. The pocket 46 can improve patient comfort and ease of accessibility to patient monitors and/or devices.

An opening, such as, but not limited to a slit 52 may be formed within the pocket 46 and may extend through the front side 22, thereby allowing access to the patient 10. The slit 52 is configured to receive at least one tube, such as a chest tube (not shown), or other device, removably coupled to a drain (not shown) removably implanted in the patient 10 following a cardiothoracic procedure so to allow for proper drainage of liquid formed as a result of the surgery. The slit 52 is sized such that it may receive the tube(s) and allow for the tube(s) to extend through the front side 22 of the body panel 14. Alternatively, the tube(s) may be received within an opening forming a flap (not shown). In exemplary embodiments, a closure mechanism 54 may be formed in the pocket 46 proximate to the slit 52 (as shown in FIG. 1) in order to maintain compressive value of the garment 12 once the tube(s) is inserted. In one exemplary embodiment, the closure mechanism 54 is formed from a hook-and-loop material such that the closure mechanism 54 is configured to releasably close the slit 52 either while the tube(s) is inserted in the slit 52 or without any tube(s). Other closure mechanisms, such as but not limited to, eye hook, snaps, buttons, or the like, may be used as the closure mechanism 54. In one exemplary embodiment, the garment 12 may include two slits 52 as shown in FIG. 3.

The rear side 24 of the body panel 14 is associated with the shoulder strap 16 at the upper edge 42 of the rear side 24 so to allow for the releasable coupling of the shoulder strap 16 and the front body 22. Illustratively, the garment 12 may include at least two shoulder straps 16, each configured to abut a portion of a wearer's shoulder (not shown). As shown in FIGS. 1 and 3, the shoulder straps 16 may extend from an area proximate to the upper edge 42 of the rear side 24 away from the rear side 24 and are configured to removably couple to the front body 22 so to secure the patient 10 in the garment 12 and maintain the desired compression value. Illustratively, the shoulder straps 16 may be coupled to the front body 22 via a hook-and-loop material or other fastener mechanism known to those skilled in the art. The front side 22 may include at least one section of the hook-and-loop material (not shown) and the shoulder strap 16 includes a matable section of hook-and-loop material (not shown) configured to engage the other mating section of material located on the front body 22. Illustratively, the hook-and-loop section of the shoulder strap 16 may extend along at least a portion of the length of the strap 16. The shoulder straps 16 are configured to adjust in length depending on the body size and body type of the patient 10. This allows for patients 10 of varying sizes to use the same size garment 12, thereby decreasing costs for caregivers and healthcare facilities.

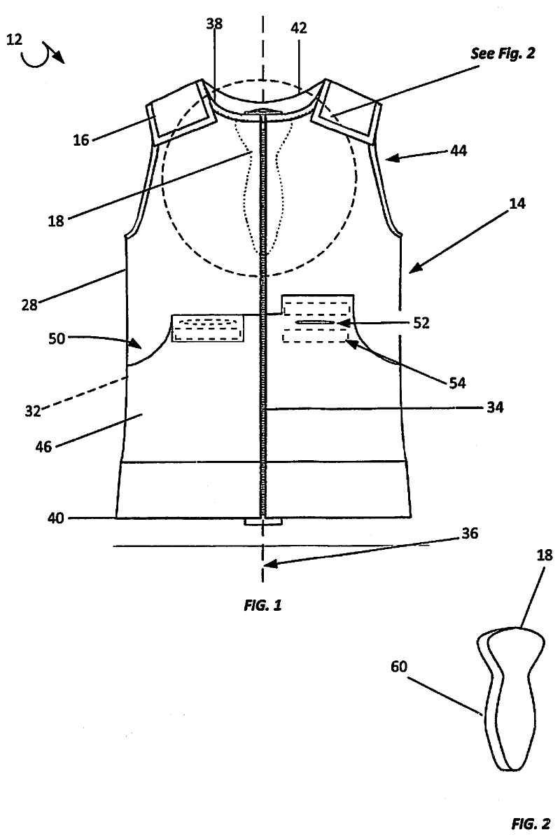

As discussed above, in exemplary embodiments the pillow 18 may be integrally formed in the front side 22 of the body panel 14 as shown in FIGS. 1 and 3. The pillow 18 may be located within the front side 22 such that it may be positioned on a patient 10 so that the pillow 18 extends generally vertically along the central axis 36 and is proximate to or abuts at least a portion of the length of the incision located on the patient's upper region 20. The pillow 18 is configured to apply pressure on the patient's incision and surrounding skin in order to provide sternal support. In exemplary embodiments, this pressure applied to the sternal area is greater than the pressure that the garment 12 would apply without the pillow 18. Since the pillow 18 extends inwardly towards the patient 10 more than the front side 22, the pillow 18 overcomes the issue of pectoral tissue in overweight patients displacing the front side 22. When the front side 22 becomes displaced, it is no longer in contact with the patient's incision and rather is located above the desired position. By using the pillow 18 as described herein, the garment 12 is advantageous in that it increases its efficaciousness regardless of the size and body mass of the patient 10. Further, the shape of the pillow 18 also acts as a deterrent against displacement of the front side 22 and the pillow 18. Illustratively, the pillow 18 is shaped to generally approximate the shape of the sternum (the sternal shape illustratively shown in FIG. 2) as shown in FIGS. 1-3. It should be appreciated that the pillow 18 may be shaped differently, such as a rectangle, circle, oval, or any other regular or irregular shape. The pillow 18 may be located generally vertically in relation to the central axis 36 and extends from above the upper edge 38 of the front side 22 downwardly towards the bottom edge 40 of the front side 22. As stated above, the pillow 18 is sized to extend along at least a portion of, or the entire, length of the patient's incision. Illustratively, the size of the pillow 18 may be comparable with the size and position of the sternum of the patient 10 such that the pillow 18 ends slightly below the natural termination point of the sternum. When placed along the incision, the pillow 18 may encounter bodily fluids of the patient. As the pillow 18 may, in exemplary embodiments, be made from closed cell foam, absorption of these fluids is avoided. In other exemplary embodiments, the pillow 18 may be made of more than one material. In exemplary embodiments, the pillow 18 may include an outer cover 60 formed from an antimicrobial and/or washable fabric.

In use, the rear side 24 is worn proximate to the rear upper region of the patient 10, the lateral sides 28 engage the side body portion (not shown) of the patient 10, the front body 22 engages the front upper region 20 of the patient 10, and the patients arms extend through the apertures 44 formed in the garment. The caregiver may then guide a tube, coupled to a drain, through the slit 52 such that it extends away from the patient 10 and is configured to collect fluid resulting from the surgery. Upon positioning the tube through the slit 52, the caregiver actuates (e.g., zips) the closure mechanism 54 so that the patient 10 is removably positioned between the front side 22, the back side 24, and the lateral sides 28. In light of the size and body mass of the patient 10, the caregiver removably couples the shoulder straps 16 to the front side 22 by extending the shoulder straps 16 upwardly around the shoulders of the patient 10 and, when engaged with the shoulders, pulling the straps 16 downwardly towards the front side 22 until the straps 16 releasably engage the front side 22. The pillow 18 is now tightly compressed against the patient's incision, and the patient 10 is free to continue with daily activities and prescribed therapies with increased comfort and expedited healing.

As used in the specification and the appended claims, the singular forms “a,” “an” and “the” include plural referents unless the context clearly dictates otherwise.

It should further be noted that any patents, applications and publications referred to herein are incorporated by reference in their entirety.

Although only a number of exemplary embodiments have been described in detail above, those skilled in the art will readily appreciate that many modifications are possible in the exemplary embodiments without materially departing from the novel teachings and advantages. Accordingly, all such modifications are intended to be included within the scope of this disclosure as defined in the following claims.

What is claimed is:

1. A compression garment for use after surgery, such as cardiothoracic surgery, comprising:
(a) a body panel configured to lie in close proximity to an upper region of a patient and formed from a compression material, the body panel including a front side configured to lie adjacent to a front upper region of the wearer, and further including a rear side configured to lie adjacent to a rear upper region of the patient;
(b) a shoulder strap associated with the rear side of the body panel and configured to extend upwardly away from the rear side and configured to removably associate with the front side of the body panel, the shoulder strap further configured to be adjustable so as to adjust the distance that the shoulder strap extends; and,
(c) a pillow integrally formed in the front side of the body panel, the pillow formed as a generally oblong shape and sized to project inwardly towards the patient, the pillow configured to apply pressure on the front upper region of the patient.

2. The compression garment of claim 1, wherein the pillow has an outline shape approximating the outline of a sternum.

3. The compression garment of claim 1, further comprising at least one pocket formed in the front side of the body panel, each pocket having a first opening.

4. The compression garment of claim 3, wherein the at least one pocket includes a second opening adapted to receive tubing.

5. The compression garment of claim 4, wherein for at least one of the at least one pockets the second opening comprises a slit.

6. A compression garment, comprising:
(a) a body panel configured to lie in close proximity to an upper region of a patient, the body panel including a front side configured to lie adjacent to a front upper region of the wearer, and a rear side configured to lie adjacent to a rear upper region of the patient;
(b) a shoulder strap coupled to the rear side of the body panel and configured to removably couple to the front side of the body panel; and,
(c) a pillow integrally formed in the front side of the body panel and configured to apply compressive pressure on the patient.

7. A compression garment for use after surgery, such as cardiothoracic surgery, comprising:
(a) a body panel configured to lie in close proximity to an upper region of a patient and formed from a compression material, the body panel comprising
i. a front side configured to lie adjacent to a front upper region of the wearer,
ii. a rear side configured to lie adjacent to a rear upper region of the patient,
iii. at least one pocket formed in the front side, and,
iv. a slit formed in the at least one pocket, the slit having an opening;
(b) a shoulder strap associated with the rear side of the body panel and configured to extend upwardly away from the rear side and configured to removably associate with the front side of the body panel, the shoulder strap further configured to be adjustable so as to adjust the distance that the shoulder strap extends; and,
(c) a pillow integrally formed in the front side of the body panel, the pillow formed as a generally oblong shape and sized to project inwardly towards the patient, the pillow configured to apply pressure on the front upper region of the patient.
